(19) United States
(12) Patent Application Publication
(10) Pub. No.: US 2010/0239672 A1
(43) Pub. Date: Sep. 23, 2010

(54) LAYER SILICATE NANOCOMPOSITES OF POLYMER HYDROGELS AND THEIR USE IN TISSUE EXPANDERS

(76) Inventors: Lajos Kemény, Szeged (HU); Imre Dékány, Szeged (HU); János Varga, Szeged (HU); László Janovák, Kunbaja (HU)

Correspondence Address: HAHN & VOIGHT PLLC, 1012 14TH STREET, NW, SUITE 620, WASHINGTON, DC, 20005, US

(21) Appl. No.: 12/602,398

(22) PCT Filed: May 30, 2008
(86) PCT No.: PCT/HU2008/000062

§ 371 Date: May 3, 2010

(30) Foreign Application Priority Data
May 31, 2007 (HU) P0700384

(51) Int. Cl. A61K 9/00; A61K 31/785

(52) U.S. Cl. 424/487; 424/78.02

(57) ABSTRACT

The invention relates to nanocomposites comprising of (i) hydrogels synthetized by copolymerization of N-isopropylacrylamide and/or acrylamide and/or acrylic acid monomers and of (ii) layer silicates, and to the process for preparing them. The invention covers osmotically active hydrogel expanders containing said nano-composites, suitable for tissue expansion and the use of said materials for obtaining live skin.

The invention relates to nanocomposites comprising of (i) hydrogels synthetized by copolymerization of N-isopropylacrylamide and/or acrylamide and/or acrylic acid monomers and of (ii) layer silicates, and to the process for preparing them. The invention also relates to osmotically active hydrogel expanders containing said nanocomposites suitable for tissue expansion, and the use of said materials for obtaining live skin.

### THE DISCLOSURE OF THE PRIOR ART

Hydrogels are cross-linked polymers having hydrophilic and hydrophobic parts in appropriate ratios, allowing them to swell in aqueous media to several times their original volume without either dissolving or changing their shape to any considerable extent. These materials are also termed “intelligent gels”, because, depending on their composition, they perceive changes in one or several environmental parameters (temperature, pH, light, magnetic field, etc.) and respond with a functional reaction (swelling, shrinking, sol-gel conversion). Owing to their advantageous properties hydrogels are widely utilized in medicine (controlled drug release, wound treatment, contact lenses) [S. R. Khetani, S, N. Bhatia, Biotechnology 17, 1-8 (2006); P. S. Keshava Murthy, Y. Murali Mohan, J. Sreeramulu, K. Mohana Raju, Reactive & Functional Polymers 63, 11-26 (2006); D. S. W. Benoita, C. R. Nuttelmana, S. D. Collinsa, K. S. Ansetha, Biomaterials 27, 6102-6110 (2006); J. P. Hervas Perez, E. Lopez-Cabarcos, B. Lopez-Ruiz, Biomolecular Engineering 23, 233-245 (2006)] as well as in other fields (environmental protection, agriculture) [D. R. Kioussis, Peter Kofinas, Polymer 46, 9342-9347 (2005); P. Liu, J. Peng, J. Li, J. Wu, Rad. Phys. and Chem. 72, 635-638 (2005)].

Hydrogels utilized in human health care (e.g. biomaterials, controlled drug delivery, electrophoretic gels) are required to swell without dissolving in the aqueous phase and to be biocompatible. Several properties of hydrogels make them suitable for health care applications and for contact with living tissues. They resemble living tissues not only in their ability to absorb large amounts of water, but also in being permeable to small molecules such as oxygen, nutrients and various metabolites. The soft, elastic material of swollen hydrogels does not irritate the neighboring tissues and cells and, due to its low surface tension attributable to its high water content, it reduces protein adsorption and denaturation. These gels are freed of undesirable intermediates, residual initiator and monomers and manufactured in a variety of shapes and sizes [N. A. Peppas, F. Giordano, P. Colombo, D. N. Robinson, C. Donini, Int. Jour. of Pharm. 245, 83-91 (2002); E. Karadag, D. Saraydin, O. Guven, Nuc. Instr. and Meth. in Phys. Res. 225, 489-496 (2004); N. A. Peppas, P. Bures, W. Leobandung, H. Ichikawa, Eur. J. Pharmacet. 50, 27 (2000); D. Saraydyn, S. U. Saraydyn, E. Karadag, E. Koptagel, O. Guven, Nuc. Instr. and Meth. in Phys. Res. 217, 281-292 (2004); I. Y. Galaev, B. Mattiasson, Trend Biotechnol. 17, 335 (1999)].

Hydrophilic monomers often used in hydrogels are acrylamide (AAm) and acrylic acid (hereinafter abbreviated as AAc). The hydrophilic character of these materials is accounted for by their amino and carboxyl groups.

Acrylamide (hereinafter abbreviated as AAm) based homo- and copolymers have an especially high water absorption capacity and oxygen permeability and are highly biocompatible [D. Saraydyn, S. U. Saraydyn, E. Karadag, E. Koptagel, O. Guven, Nuc. Instr. and Meth. in Phys. Res. 217, 281-292 (2004); O. Guven, M. Sen, E. Karadag, D. Saraydin, Radiat. Chem. Phys. 56, 381 (1999)].

The surgical application of hydrogels containing AAm homo- and copolymers is the subject of numerous patents. These have mainly been utilized for implantation, as described e.g. in Hungarian patent application HUO302054, Bulgarian patent specification BG101251, U.S. patent application US2005175704 and international publication document WO03084573.

In September 2003, Novaes and Berg carried out an extensive study on the poly(AAm)-based product Aquamid® [Wilse de Cassia Novaes, Agnes Berg, Aesthetic Plastic Surgery, 27, 276-300 (2003)]. In the course of the test, 59 patients with mostly labial, nasal, facial and mental injuries were subjected to correctional surgery. The patients were 20 to 60 years of age and were typically followed up for 9 months. The results showed that the material tested is biocompatible, non-toxic, non-inflammatory and non-metabolized.

The high molecular weight poly(AAc) is a bioadhesive polymer capable of adhering to the mucous cells in the eyes, the nose, the lungs, the intestinal tract or the vagina. It is therefore widely used as a drug carrier in the field of controlled drug release, because by adhering to the cells it increases the residence time of the drugs in the cells [E. S. Ron, L. Bromberg, S. Luczak, M. Kearney, D. Deaver, M. Schiller, Smart hydrogel: a novel mucosal delivery system, Proc. Int. Symp. Control. Rel. Bioact. Mater. 24, 407-408 (1997); E. S. Ron, E. J. Roos, A. K. Staples, L. E. Bromberg, M. E. Schiller, Interpenetrating polymer networks for sustained dermal delivery, Proc. Int. Symp. Control. Rel. Bioact. Mater. 23, 128-129 (1996); A. M. Potts, S. Jackson, N. Washington, P. Gilchrist, E. S. Ron, M. Schiller, C. G. Wilson, In vivo determination of the esophageal retention of smart hydrogel, Proc. Int. Symp. Control. Rel. Bioact. Mater. 24, 335-336 (1997)].

In U.S. Pat. No. 5,013,769, AAc homopolymer- and copolymer-based hydrogels were utilized as wound dressing material and for covering the skin.

There has recently been increased interest in thermosensitive hydrogels in the field of medical applications. One of the most intensively investigated materials employed in these hydrogels is poly(N-isopropylacrylamide) [hereinafter abbreviated as poly(NIPAAm)]. The thermosensitive properties of poly(NIPAAm) have been extensively studied and modelled [K. S. Chen, J. C. Tsai, C. W. Chou, M. R. Yang, J. M. Yang, Materials Science and Engineering 20, 203-208 (2002); Andras Szilagyi, Miklos Zrinyi, Polymer 46, 10011-10016 (2005); M. R. Guilherme, G. M. Campesea, E. Radovanovic, A. F. Rubira, E. B. Tambourgi, E. C. Muniz, Journal of Membrane Science 275, 187-194 (2006); D. C. Coughlan, O. I. Corrigan. Intern. Journal of Pharmaceutics 313, 163-174, (2006); V. Kumar, C. V. Chaudhari, Y. K. Bhardwaj, N. K. Goel, S. Sabharwal. Eur. Pol. Jour. 42, 235-246, (2006)]. These materials collapse in aqueous phase at about 32° C.; at lower temperatures, however, they are capable of considerable swelling.

Han, Bae et al. studied the application of N-isopropylacrylamide-acrylic acid copolymer [hereinafter abbreviated as poly(NIPAAm-co-AAc)] as artificial pancreas [Y. H. Bae, B. Vernon, C. K. Han, S. W. Kim, Extracellular matrix for a rechargeable cell delivery system, J. Control. Release 53, 249-258 (1998)]. After transplantation, islets of Langerhans often aggregated, which caused necrosis. The cells were therefore placed to the above mentioned thermosensitive solution prior to implantation into diabetic patients. The solution provided a kind of immunoprotection for the cells.

Research aiming at the preparation of gels comprising various inorganic fillers has also been performed and the properties of gels have been shown to be considerably altered by fillers N. Alexandre, P. Dubois. Mat. Science and Engineering, 28, 1-63 (2000); S. Sinha Ray, M. Bousmina. Prog. in Mat. Science 50, 962-1079 (2005); J. M. Yeh, S. J. Liou, Y. W. Chang. Jour. of App. Poly. Sci., 91, 3489-3496 (2004); X. Xia, J. Yih, N. A. D'Souza, Z. Hu. Polymer 44, 3389-3393 (2003); Y. Xiang, Z. Peng, D. Chen, European Polymer Journal 42, 2125-2132 (2006); N. A. Churochkina, S. G. Starodoubtsev, A. R. Khokhlov. Poly. Gels and Netw. 6, 205-215 (1998)].

For example, the application of clay-containing polymer hydrogels for therapeutic purposes has been described in Japanese patent applications J2004091755 and JP2005290072. For example, Japanese patent application JP2005290073 relates to hydrogels comprising poly(NIPAAm) and clay.

The improvement of the physical properties of polymers by the addition of layer silicates is also well-known in the technical literature. For example, Don and Feng synthetized a polylactic acid-based composite comprising Na-montmorillonite (hereinafter abbreviated as Na-mont), which they successfully used for controlled drug release [Yuancai Dong, Si-Shen Feng, Biomaterials 26, 6068-6076 (2005)] and experimentally proved that the montmorillonite content of the hydrogel increased the uptake of the model active substance (Coumarin 6) by the cells used.

In the course of time there have been a number of various methods tested for obtaining skin for closing different defects. In 1957, Neuman used a balloon placed in a retroauricular position in order to expand the tissues and the skin for ear reconstruction.

Nearly 20 years later, in 1976 Radován was the first to use subcutaneous silicone tissue expander for breast remodeling. The popularity of this method has been undiminished for a very long time, even though its applicability is countered by numerous disadvantages. Due to the special geometry of the filling valve and the balloon, the expander is very often damaged. In addition, in cases of application for skin expansion the skin covering the filling valve has to be punctured at the time of every fill-up, causing pain. In the case of children the fear of pain and, consequently, of fill-ups is distinctly disadvantageous. The patient has to present for control visits on a regular basis, which is costly and time-consuming. The need for an alternative method for skin expansion and an expander lacking the above mentioned disadvantages of traditional expanders has long been recognized. Attention has turned to intelligent nanocolloids and these materials have increasingly been employed for this role.

The groundwork for osmotic expanders was laid by Prof. Dr. Wiese in the nineteen-nineties. He achieved tissue expansion by using an active hydrogel system. The idea is based on two factors, namely (i) the physiological fact that human tissues consist mostly of water, and (ii) the phenomenon of osmosis, well-known in plants, which are capable of generating high hydrostatic pressures. The osmotic system can exert sufficient pressure and transport adequate amounts of fluid to attain the appropriate tissue pressure. As a result of swelling, the expanded mass/area of the skin increases.

The advantages of the application of osmotic expanders are the following:
the implanted expander is extremely small,
a very small aperture (incision) is needed for implantation, which means a minimal surgical trauma,
there is no need for regular fill-ups, therefore there is less pain and fear for children,
less post-operative control means time-saving for the patient,
there is a lower risk of the infection of the implant,
the patient is spared the constant discomfort of tightness associated with fill-ups,
it is fast, simple and reliable.

In U.S. Pat. No. 5,496,368 Dr. Wiese performed tissue expansion for forming a cavity to receive an implant and for obtaining tissue suitable for self-transplantation, and used methylmethacrylate-N-vinylpyrrolidone copolymer based hydrogel and its saponified derivative. This material, i.e. N-vinylpyrrolidone methacrylate had earlier been used in contact lenses and its non-toxicity had been proven by testing. One of the two hydrogel types described in Dr. Wiese's above mentioned US patent swelled to about ten times its original volume, but lost its mechanical and shape stability in the process and was therefore encapsulated in a semipermeable membrane. The shape stability of the other hydrogel was appropriate, but it swelled to no more than 3.6 times its original volume.

The object of our work was to develop an expander of the osmotic hydrogel type with good mechanical and shape stability that undergoes considerable swelling under the effect of osmotic forces when placed in aqueous medium, while retaining its original shape. When such material is implanted under the skin of the patient, it swells to many times its original volume by uptaking the interstitial fluid of the surrounding tissues, expanding the overlapping skin in the process. The excess skin obtained in this way can later be utilized in plastic surgical procedures.

This object was achieved by the development of a hydrogel nanocomposite comprising N-isopropylacrylamide, acrylamide and/or acrylic acid based polymers and a filler of the layer silicate type.

### SUMMARY OF THE INVENTION

The invention relates to nanocomposites comprising (i) hydrogels synthetized by homo- or copolymerization of N-isopropylacrylamide, acrylamide and/or acrylic acid monomers in the presence of crosslinkers and (ii) a layer silicate filler.

The invention also relates to the preparation of said nanocomposite, in the course of which the monomers and other polymerization components, namely the crosslinker, the initiator and the accelerator are added to the filler dispersed in distilled water, and anionic radical polymerization is carried out.

The invention also relates to an osmotically active tissue expander comprising the nanocomposite according to the invention.

The invention also relates to the use of the expander according to the invention to expand the skin of living organisms and to obtain skin suitable for the repair of live skin.

In a preferred embodiment the nanocomposite comprises a polymeric hydrogel comprising

0 to 90 mol % of N-isopropylacrylamide monomer and 100 to 10 mol % of acrylamide monomer, or

0 to 30 mol % of N-isopropylacrylamide monomer and 100 to 70 mol % of acrylic acid monomer, or

0 to 100 mol % of acrylamide monomer and 100 to 0 mol % of acrylic acid monomer.

In another preferred embodiment, the layer silicate filler is sodium montmorillonite (hereinafter abbreviated as Na-mont) or organophilized montmorillonite, where the organophilized montmorillonite is Na-montmorillonite modified by amines with carbon chains of various lengths (CₙH₂ₙ₊₁—NH₂), (n=4, 8, 12, 16, 18) (hereinafter abbreviated as C₄-, C₈-, C₁₂-, C₁₆-, C₁₈-mont).

The amount of the filler relative to the total dry mass of the nanocomposite is preferably between 0.1 and 10 wt %.

The procedure according to the invention preferably employs N,N-methylene-bisacrylamide (BisAAm) as crosslinker, potassium persulfate (KPS) as initiator and N,N,N′,N′-tetramethylethylenediamine (TEMED) as accelerator. The crosslinker is preferably used in a molar ratio of 50 to 1500 relative to the amount of monomer(s). Sulfate anion radicals for the polymerization are supplied by the KPS-TEMED redox pair.

### DETAILED DESCRIPTION OF THE INVENTION

The nanocomposites according to the invention comprise AAm or AAc homopolymer or a copolymer comprising NIPAAm, AAm and/or AAc monomers at various ratios, which copolymer is always built up from two of the above-mentioned monomers. Thus, NIPAAm-AAm, NIPAAm-AAc and AAm-AAc based copolymers are prepared [poly(NIPAAm-co-AAm), poly(NIPAAm-co-AAc) and poly(AAm-co-AAc)].

In the course of the preparation of the nanocomposites according to the invention, the filler is dispersed in distilled water, the monomer(s) and the other components listed above are added to the dispersion and the reaction is performed in test tubes at a temperature of 40-60° C., in nitrogen atmosphere. The hydrogel obtained in this way is cut up and dried, in the course of which it shrinks to 1/40 its original size.

To purify the hydrogel nanocomposite obtained, it is reswollen and soaked for a fixed period of time to remove starting materials and other contaminations. The reswollen sample regains the original size and shape it had before drying. It is then dried again, when it acquires the form suitable for implantation.

The three-dimensional gel structure is presented in FIG. 1. For the sake of simplicity, the figure only shows a NIPAAm-based network; the polymer structure is similar in the case of all three starting monomers.

Montmorillonite, which is used as a filler (Al₂(OH)₂Si₄O₁₀), is a member of the group of phyllosilicates (layer silicates). Numerous substitutions can be made its theoretical formula; water and other molecules can be incorporated into its structural layers. The extensive swelling of montmorillonite-containing clays is the consequence of the presence of water. Characteristically, three oxygen atoms of the [SiO₄]⁴⁻ tetrahedrons are shared by the neighboring equiplanar tetrahedrons, as shown in FIG. 2. Layers having theoretically infinite dimensions are thus formed, which layers are interlinked through cations bond to the remaining charge. The intralayer bonding is strong (ionic, covalent), whereas the interlayer bonding is considerably weaker (van der Waals bond), therefore the layers easily divide from each other and thus, these minerals easily split parallel with the plane of the layers. Their structure is built up by three types of layers, with alternating tetrahedron layers, octahedron layers and layers with large excess negative charge. The excess negative charge created by Al³⁺ substitution in the tetrahedron layer and Mg²⁺ or Fe²⁺ substitution in the octahedron layer are counterbalanced by interlaminar Na⁺ and Ca²⁺ ions. These minerals therefore characterized by ion exchanging capability. In organophilized montmorillonite, amines delaminate the silicate blocks to different extents depending on the length of the carbon chain, as it is shown in FIG. 3.

### DESCRIPTION OF THE FIGURES

A brief description of the enclosed Figures follows below.

FIG. 1 shows the gel structure formed by NIPAAm monomer with bisacrylamide as crosslinker.

FIG. 2 shows the structure of montmorillonite.

FIG. 3 shows the penetration of carbon chains having of 4, 12 and 18 carbon atoms substituted by an amino group among the montmorillonite layers, and the resulting structure of hydrophobized Na-montmorillonite.

In FIG. 4 the swelling of poly(NIPAAm-co-AAm) copolymers of various compositions is compared in distilled water at 25-40° C.

FIG. 5 the swelling of poly(NIPAAm-co-AAc) copolymers of various compositions is compared in distilled water at 25-40° C.

FIG. 6 the swelling of poly(AAm-co-AAc) copolymers of various compositions is compared in distilled water at 25-40° C.

FIG. 7 shows the XRD curve of a typical intercalation structure in a poly(NIPAAm-co-AAm) copolymer based composite containing 25 wt % C₄-montmorillonite as filler.

FIG. 8 shows the XRD curve of a typical exfoliation structure for a poly(NIPAAm)-based composite containing 25 wt % C₄-montmorillonite as filler.

FIG. 9 shows the effect of Na-montmorillonite filler on gel swelling.

FIG. 10 shows the effect of C₄-montmorillonite filler on gel swelling.

FIG. 11 shows the effect of C₁₂-montmorillonite filler on gel swelling.

FIG. 12 shows the effect of 18-montmorillonite filler on gel swelling.

FIG. 13 shows the electrolyte sensitivity of gels, i.e. the effect of electrolyte concentration on the swelling of composite gels.

FIG. 14 shows the temperature dependence of polymer swelling.

FIG. 15 shows the effect of filler concentration on the mechanical properties of gels.

FIG. 16 shows the effect of the monomer/crosslinker ratio on the swelling of poly(AAm) gel.

FIG. 17 shows the effect of the monomer/crosslinker ratio on the swelling of poly(AAc) gel.

FIG. 18 shows the swelling kinetics of poly(AAm-co-AAc) copolymer containing C₁₂-montmorillonite filler in physiological saline at 36.5° C.

FIG. 19 shows the swelling kinetics of implanted gels shown in FIGS. 21 to 23 under in vitro conditions.

FIG. 20 shows schematic representation of gel swelling.

FIG. 21 shows Poly(NIPAAm-co-AAm) gel containing 1 wt % Na-montmorillonite in swollen and dried state.

FIG. 22 shows Poly(AAc) gel containing 5 wt % Na-montmorillonite in swollen and dried state.

FIG. 23 shows Poly(AAm-co-AAc) gel containing 5 wt % Na-montmorillonite in swollen and dried state.

FIG. 24 shows the implantation site in a rat after implantation.

FIGS. 25 to 27 show the process of swelling.

FIGS. 28 to 33 shows the surgery site and the excised samples.

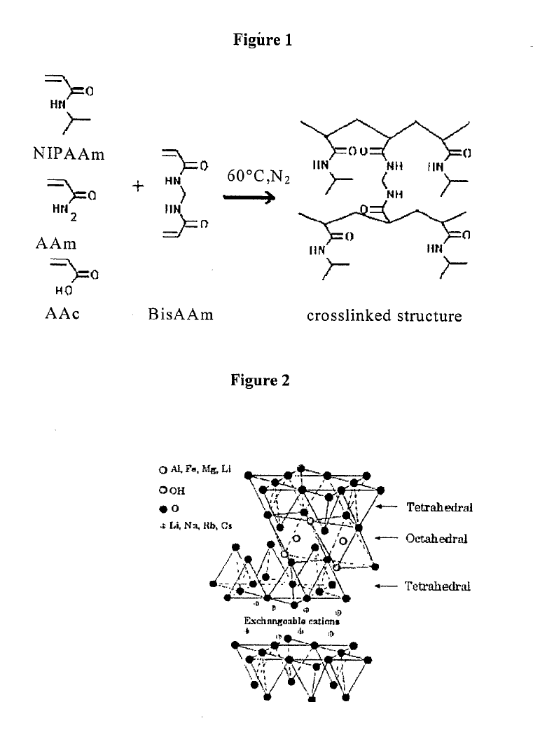

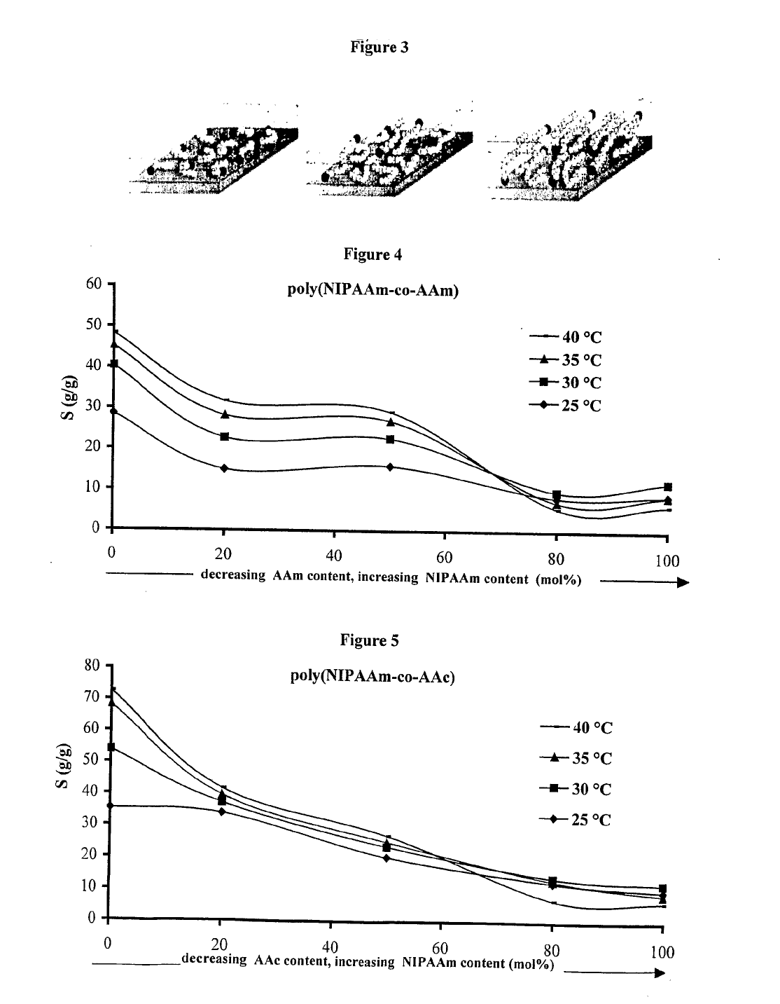

### EXPERIMENTAL SECTION

### Abbreviations

NIPAAm: N-isopropylacrylamide, AAm: acrylamide, AAc: acrylic acid, BisAAm: N,N-methylenebisacrylamide, KPS: potassium persulfate, TEMED: N,N,N′,N′-tetramethylethylenediamine, poly(NIPAAm): polymer synthetized of NIPAAm monomer, poly(AAm): polymer synthetized of AAm monomer, poly(AAc): polymer synthetized of AAc monomer, poly(NIPAAm-co-AAm): copolymer synthetized of NIPAAm and AAm monomers, poly(NIPAAm-co-AAc): polymer synthetized of NIPAAm and AAc monomers, poly(AAm-co-AAc): copolymer synthetized of AAm and AAc monomers, Na-mont: Na-montmorillonite, C₄-mont: Na-montmorillonite organophilized with an amine having a C₄ carbon chain, C₁₂-wont: Na-montmorillonite organophilized with amine having a C₁₂ carbon chain, C₁₈-mont: Na-montmorillonite organophilized with amine having a C₁₈ carbon chain.

### Comparative Example 1

### Synthesis of 100% AAm-Based Polymer [poly(AAm)] Hydrogel

2.5 mol/l AAm monomer stock solution and 0.1 mol/l BisAAm crosslinker stock solution are prepared in distilled water. 4 ml of monomer stock solution (0.7108 g) and 0.5 ml of crosslinker stock solution (7.7085*10⁻³ g) are added to a test tube, thereby setting the monomer/crosslinker ratio to 200. To this solution, 1.25*10⁻⁴ g of KPS (initiator) and 7.75*10⁻³ g of TEMED (accelerator) are added and the solution obtained is filled up to 10 ml with distilled water. The test tube is flushed with N₂ for 3 to 5 min, closed air-tight and placed in a 50-60° C. water bath for half an hour. After the completion of the polymerization the gel obtained is removed from the test tube, cut into pieces with a scalpel and dried to constant weight in a drying oven at 70-80° C. for 3 to 4 days.

### Comparative Example 2

### Synthesis of 100% AAc-Based Polymer [poly(AAc)] Hydrogel

2.5 mol/1 monomer stock solution (AAc) and 0.1 mol/l crosslinker stock solution (BisAAm) are prepared in distilled water. 4 ml of monomer stock solution (0.7206 g) and 0.5 ml of crosslinker stock solution (7.7085*10⁻³ g) are added to a test tube, thereby setting the monomer/crosslinker ratio to 200. To this solution, 1.25*10⁻⁴ g of KPS (initiator) and 7.75*10⁻³ g of TEMED (accelerator) are added and the solution obtained is filled up to 10 ml with distilled water. The test tube is flushed with N₂ for 3 to 5 min, closed air-tight and placed in a 50-60° C. water bath for half an hour. After the completion of the polymerization the gel obtained is removed from the test tube, cut into pieces with a scalpel and dried to constant weight in a drying oven at 70-80° C. for 3 to 4 days.

### Comparative Example 3

### Synthesis of 50% AAm+50% AAc Based Polymer [poly(AAm-co-AAc)] Hydrogel

2.5 mol/1 monomer stock solutions (AAm and AAc) and 0.1 mol/l crosslinker stock solution (BisAAm) are prepared in distilled water. 2 ml of each monomer stock solution (0.3554 g of AAm and 0.3603 g of AAc) and 0.5 ml of crosslinker stock solution (7.7085*10⁻³ g) are added to a test tube, thereby setting the monomer/crosslinker ratio to 200. To this solution, 1.25*10⁻⁴ g of KPS (initiator) and 7.75*10⁻³ g of TEMED (accelerator) are added and the solution obtained is filled up to 10 ml with distilled water. The test tube is flushed with N₂ for 3 to 5 min, closed air-tight and placed in a 50-60° C. water bath for half an hour. After the completion of the polymerization the gel obtained is removed from the test tube, cut into pieces with a scalpel and dried to constant weight in a drying oven at 70-80° C. for 3 to 4 days.

### Example 4

### Synthesis of Organophilized Montmorillonite

The amine is dissolved in acidified ethanol-water mixture and added to Na-montmorillonite pre-swollen in distilled water at a ratio of 100 meq/g; the system is next stirred for 24 hours. After the completion of the ion exchange the suspensions are centrifuged and filtered. The hydrophobized filler obtained in this way is dried and ground to a particle size of 200 μm.

### Example 5

### Synthesis of 100% AAm-Based Hydrogel Nanocomposite Containing 5 wt % C₄-Montmorillonite

2.5 mol/l AAm monomer stock solution and 0.1 mol/l BisAAm crosslinker stock solution are prepared in distilled water. 4 ml of monomer stock solution (0.7108 g) and 0.5 ml of crosslinker stock solution (7.7085*10⁻³ g) are added to a test tube, thereby setting the monomer/crosslinker ratio to 200. 0.03823 g of C₄-montmorillonite is dispersed in 5 ml distilled water and the dispersion obtained is added to the previously prepared monomer/crosslinker solution. Finally, 1.25*10⁻⁴ g of KPS (initiator) and 7.75*10⁻³ g of TEMED (accelerator) are added to this solution and the solution obtained is filled up to 10 ml with distilled water. The test tube is flushed with N₂ for 3 to 5 min, closed air-tight and placed in a 50-60° C. water bath for half an hour. After the completion of the polymerization the composite obtained is removed from the test tube, cut into pieces with a scalpel and dried to constant weight in a drying oven at 70-80° C. for 3 to 4 days.

Prior to in vivo experiments the samples are reswollen in distilled water and stored under these conditions for a minimum of one week with continuous renewal of water, thereby removing the unreacted monomers and other contaminations (residual initiator and accelerator, etc.) from the polymer skeleton.

Note: the value of filler concentration (5 wt %) refers to the mass of the completely dried composite.

### Example 6

### Synthesis of 100% AAc-Based Hydrogel Nanocomposite Containing 5 wt % C₁₂-Montmorillonite

2.5 mol/1 monomer stock solution (AAc) and 0.1 mol/l crosslinker stock solution (BisAAm) are prepared in distilled water. 4 ml of monomer stock solution (0.7206 g) and 0.5 ml of crosslinker stock solution (7.7085*10⁻³ g) are added to a test tube, thereby setting the monomer/crosslinker ratio to 200. 0.03875 g of C₁₂-montmorillonite is dispersed in 5 ml of distilled water and the dispersion obtained is added to the previously prepared monomer/crosslinker solution. Finally, 1.25*10⁻⁴ g of KPS (initiator) and 7.75*10⁻³ g of TEMED (accelerator) are added to this solution and the solution obtained is filled up to 10 ml with distilled water. The test tube is flushed with N₂ for 3 to 5 min, closed air-tight and placed in a 50-60° C. water bath for half an hour. After the completion of the polymerization the composite obtained is removed from the test tube, cut into pieces with a scalpel and dried to constant weight in a drying oven at 70-80° C. for 3 to 4 days.

Note: the value of filler concentration (5 wt %) refers to the mass of the completely dried composite.

### Example 7

### Synthesis of 50% NIPAAm and 50% AAm Based Hydrogel Nanocomposite Containing 1 wt % Na-Montmorillonite

2.5 mol/1 monomer stock solutions (NIPAAm and AAm) and 0.1 mol/l crosslinker stock solution (BisAAm) are prepared in distilled water. 2 ml of each monomer stock solution (0.5658 g of NIPAAm and 0.3554 g of AAc) and 0.5 ml of crosslinker (7.7085*10⁻³ g) are added to a test tube, thereby setting the monomer/crosslinker ratio to 200. 0.009463 g of Na-montmorillonite is dispersed in 5 ml of distilled water and the dispersion obtained is added to the previously prepared monomer/crosslinker solution. Finally, 1.25*10⁻⁴ g of KPS (initiator) and 7.75*10⁻³ g of TEMED (accelerator) are added to this solution and the solution obtained is filled up to 10 ml with distilled water. The test tube is flushed with N₂ for 3 to 5 min, closed air-tight and placed in a 50-60° C. water bath for half an hour. After the completion of the polymerization the composite obtained is removed from the test tube, cut into pieces with a scalpel and dried to constant weight in a drying oven at 70-80° C. for 3 to 4 days.

Note: the value of filler concentration (1 wt %) refers to the mass of the completely dried composite.

### Example 8

### Synthesis of 100% AAm-Based Hydrogel Nanocomposite Containing 5 wt % C₁₂-Montmorillonite

2.5 mol/1 monomer stock solution (AAm) and 0.1 mol/l crosslinker stock solution (BisAAm) are prepared in distilled water. 4 ml of monomer stock solution (0.7108 g) and 0.5 ml of crosslinker stock solution (7.7085*10⁻³ g) are added to a test tube, thereby setting the monomer/crosslinker ratio to 200. 0.03875 g of C₁₂-montmorillonite is dispersed in 5 ml of distilled water and the dispersion obtained is added to the previously prepared monomer/crosslinker solution. Finally, 1.25*10⁻⁴ g of KPS (initiator) and 7.75*10⁻³ g of TEMED (accelerator) are added to this solution and the solution obtained is filled up to 10 ml with distilled water. The test tube is flushed with N₂ for 3 to 5 min, closed air-tight and placed in a 50-60° C. water bath for half an hour. After the completion of the polymerization the composite obtained is removed from the test tube, cut into pieces with a scalpel and dried to constant weight in a drying oven at 70-80° C. for 3 to 4 days.

1 g of the dried sample now contains the following components:

939.8 mg of AAm, 10.19 mg of BisAAm and 50 mg of C₁₂-montmorillonite.

### Example 9

### Synthesis of 50% NIPAAm and 50% AAm Based Hydrogel Nanocomposite Containing 1 wt % Na-Montmorillonite

2.5 mol/1 monomer stock solutions (NIPAAm and AAm) and 0.1 mol/l crosslinker stock solution (BisAAm) are prepared in distilled water. 2 ml of each monomer stock solution (0.5658 g of NIPAAm and 0.3554 g of AAc) and 0.5 ml of crosslinker (7.7085*10⁻³ g) are added to a test tube, thereby setting the monomer/crosslinker ratio to 200. 0.009463 g of Na-montmorillonite is dispersed in 5 ml of distilled water and the dispersion obtained is added to the previously prepared monomer/crosslinker solution. Finally, 1.25*10⁻⁴ g of KPS (initiator) and 7.75*10⁻³ g of TEMED (accelerator) are added to this solution and the solution obtained is filled up to 10 ml with distilled water. The test tube is flushed with N₂ for 3 to 5 min, closed air-tight and placed in a 50-60° C. water bath for half an hour. After the completion of the polymerization the composite obtained is removed from the test tube, cut into pieces with a scalpel and dried to constant weight in a drying oven at 70-80° C. for 3 to 4 days.

1 g of the dried sample now contains the following components:

603 mg of NIPAAm, 378.8 mg of AAm, 8.2 mg of BisAAm and 10 mg of Na-montmorillonite.

Examination of the Gels

The swelling characteristics of the nanocomposites according to our invention were studied as described in the literature [S. Sinha Ray, M. Bousmina. Prog. in Mat. Science 50, 962-1079 (2005)].

The extent of swelling was determined gravimetrically, based on the following relationship:

D=(Sₛ−S_{d})/S_{d}[g/g]

where Sₛ is the mass of the sample in swollen and S_{d} in dried state, thus the result obtained by this relationship is the amount of water bound by unit mass of polymer. In the course of the measurement the sample was removed from the water bath of given temperature, the water on its surface was blotted, its mass was determined and it was returned to the water bath. In vitro swelling tests of the gels were carried out in distilled water, in the temperature range of 25-40° C. In the course of kinetic measurements the samples were swollen in physiological saline at 36.5° C. Since—for practical reasons—part of the tests were run at room temperature in distilled water, the temperature and ionic strength dependence of gel swelling was also examined in order to allow conclusions to be drawn regarding their expected behavior in vivo. If not stated otherwise, the monomer ratio of copolymers studied was 50/50 mol %.

The composites were subjected to X-ray diffraction (XRD) analysis in powder form. To obtain powder, the samples were completely dried and pulverized. The measurement was performed in a Philips PW diffractometer [generator: PW 1830; goniometer: PW 1820; detector: PW 1711), Cu—Kα radiation (λ=0.154 nm) 40 kV and 35 mA]. The diffraction of samples was studied in the angle range of 0-15° as described in the literature [Y. Xiang, Z. Peng, D. Chen, European Polymer Journal 42, 2125-2132 (2006); N. A. Churochkina, S. G. Starodoubtsev, A. R. Khokhlov. Poly. Gels and Netw. 6, 205-215 (1998)]. Our X-ray diffraction (XRD) results showed that our gel products were intercalation and exfoliation composites [Wen-Fu Lee, Yung-Chu Chen. European Polymer Journal 42, 1634-1642 (2006); B. Smarsly, G. Garnweitner, R. Assink, C. Jeffrey Brinker. Progress in Organic Coatings 47, 393-400 (2003)].

The Effect of Copolymer Composition on the Swelling Ratio

The effect of the individual starting monomers of various hydrophilicities on swelling of the polymers was studied. The equilibrium swelling values of polymers with various monomer compositions are listed in Table 1.

TABLE 1
Composition and equilibrium swelling of hydrogel polymers

| | | | | Equilibrium | |
|---|---|---|---|---|---|
| | | | | swelling in | |
| | | AAm | | distilled water | |
| | NIPAAm | (mol | AAc | [D(g/g)] | |
| polymer sample | (mol %) | %) | (mol %) | 25° C. | 35° C. |
| poly(NIPAAm) | 100 | 0 | 0 | 12 | 7 |
| poly(NIPAAm-co-AAm) | 50 | 50 | 0 | 16 | 28 |
| poly(AAm) | 0 | 100 | 0 | 30 | 46 |
| poly(NIPAAm-co-AAc) | 50 | 0 | 50 | 20 | 28 |
| poly(AAc) | 0 | 0 | 100 | 36 | 79 |
| poly(AAm-co-AAc) | 0 | 50 | 50 | 109 | 192 |

The table shows that in the case of copolymers in which the hydrophobic NIPAAm monomer was copolymerized with the hydrophilic AAm or AAc monomer, the extent of the swelling increases with increasing hydrophilic monomer content and the most extensive swelling is achieved by the copolymerization of the two hydrophilic monomers. FIGS. 4 and 5 demonstrate that at relatively high hydrophilic monomer (AAm or AAc) contents (in excess of 65-70%) the swelling of the gels continuously increased with increasing temperature. At relatively high NIPAAm contents (in excess of 60-70%), however, the thermosensitivity of the monomer manifested itself: at temperatures over 30° C., swelling of the samples decreased.

In the case of the NIPAAm-AAm copolymers (FIG. 4) swelling was the most extensive in the AAm/NIPAAm range of 100/0-80/20, whereas in the molar ratio range of 20/80-50/50 it remained linear. In the next molar ratio range of 50/50-20/80, gel swelling decreased considerably, and starting from 70% NIPAAm content the thermosensitive effect of NIPAAm became determinant: these samples swelled twice as extensively at 25 or 30° C. than at higher temperatures.

In the case of the NIPAAm-AAc based copolymers, again swelling was the most extensive in the molar ratio range of AAc/NIPAAm=100/0-80/20 (FIG. 5) and decreased in a linear fashion starting from the AAc/NIPAAm molar ratio of 80/20. The thermosensitive effect of the NIPAAm monomer again became determinant at a NIPAAm-content of 70 mol %.

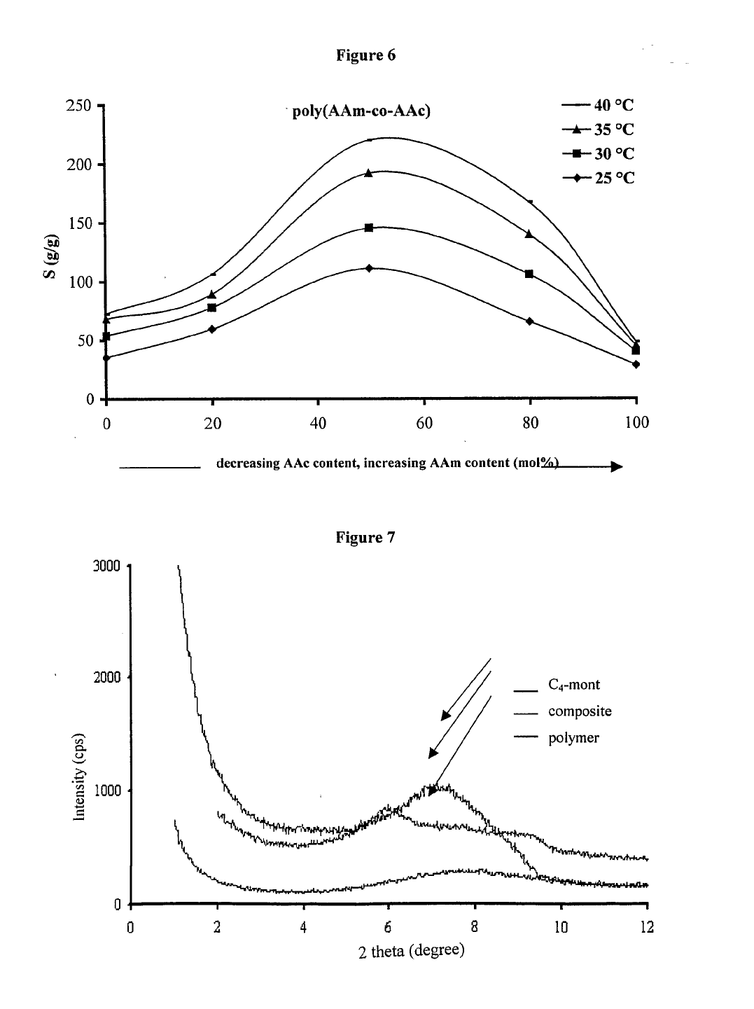

Swelling of poly(AAm-co-AAc) samples obtained by copolymerization of the two hydrophilic monomers AAm and AAc was also analyzed as a function of composition and it was established that gels containing AAm and AAc monomers in a molar ratio of 50/50 mol % swelled the most extensively, as shown in FIG. 6. The curves run parallel courses all the way, and the extent of swelling increases with increasing temperature.

Based on the above, it is expected that (i) homopolymers of the monomers AAm and AAc, (ii) their copolymers containing these monomers in any ratio, furthermore, from copolymers of these monomers with the monomer NIPAAm, (iii) NIPAAm-AAm-1-copolymers with monomer compositions between 0/100 and 90/10 and (iv) NIPAAm-AAc copolymers with monomer compositions between 0/100 and 30/70 can be utilized to advantage in nanocomposites according to the invention.

X-Ray Diffraction Analysis on the Structures of Polymer Gel Composites Containing Na-Montmorillonite or Organophilized Montmorillonite

Composites containing layer silicates are classified to three groups according to their composition (layer silicate, organic cation and polymer matrix) and their synthesis.

When polymer chains cannot penetrate among the silicate layers, phase separation composites are obtained, whose properties resemble those of traditional microcomposites.

In addition to this classical group, the so-called nanocomposites can be assigned to two types. When one or more polymer chains penetrate among the layers, but the layers still retain their parallel arrangement, an intercalation composite with a well-ordered structure is obtained.

When, however, the layers are fully and uniformly dispersed in the polymer matrix, the product of the synthesis is an exfoliation composite [M. Alexandre, P. Dubois. Mat. Science and Engineering, 28, 1-63 (2000)].

XRD measurements are suitable for the characterization of these nanostructures [Wen-Fu Lee, Yung-Chu Chen. European Polymer Journal 42, 1634-1642 (2006); B. Smarsly, G. Garnweitner, R. Assink, C. Jeffrey Brinker. Progress in Organic Coatings 47, 393-400 (2003)]. In the course of our measurements, the XRD-curve of the composite with the highest filler content (25%) was recorded in all cases.

The results of our XRD measurements reveal that the polymer chains penetrated among the layers in the course of synthesis and delaminated the silicate blocks; when the layers retained their parallel arrangement and only the interlamellar distance increased, intercalation composites were obtained. This structure is characteristic of e.g. the poly(NIPAAm-co-AAm) nanocomposite containing 25 wt % C₄-mont filler.

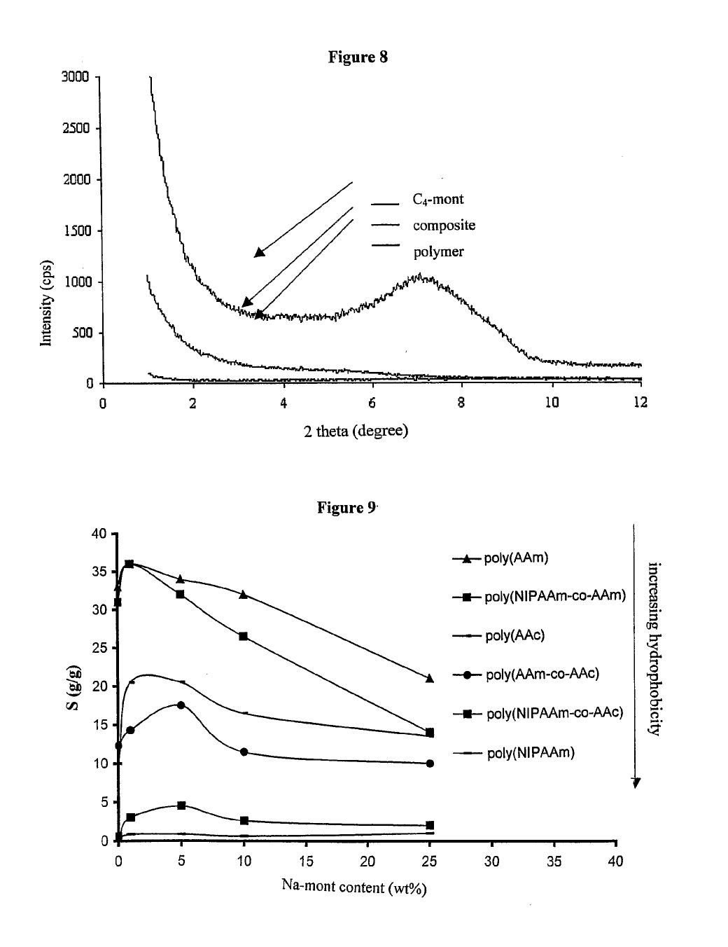

In this case the diffraction peak is shifted towards smaller angle ranges, as shown in FIG. 7. When the layers did not retain their parallel arrangement, but were totally dispersed in the polymer matrix, an exfoliation structure was formed, which is presented in FIG. 8. For example, the poly(NIPAAm) nanocomposite containing 25 wt % C₄-mont has this type of structure.

Irrespective of the hydrophilicities of filler and polymer, the synthesis of composites according to the invention resulted in intercalation or exfoliation structures in every case, which means that filler lamellae are well dispersed in the polymer skeleton.

The Effect of Fillers on the Swelling of Copolymers

In the course of the synthesis of organophilized montmorillonite fillers, amines with carbon chains of various lengths were used, which penetrated among the layers during cation exchange and delaminated them to various extents depending on the length of the carbon chain. Thus, after the completion of the reaction, fillers with different hydrophilicities were obtained: the most hydrophilic of these was Na-montmorillonite, followed by C₁₈, C₁₂ and C₄-montmorillonite.

The relationship between the hydrophilicity of the filler and the swelling of the sample was investigated in nanocomposites containing various fillers, i.e. a comparison of the swelling characteristics of nanocomposites containing Na-montmorillonite with those containing organophilized montmorillonite was carried out.

Swelling of the composite containing Na-montmorillonite as a function of filler content is presented in FIG. 9. Polymers swelling the most extensively are those containing hydrophilic AAm or AAc as starting monomer.

The experiment described above allows to establish that the presence of low concentrations of Na-montmorillonite improve the swelling characteristics of the samples. In general, samples with 1-5% filler content swell better than gels without filler; however, high filler concentrations are not advantageous from the point of view of swelling characteristics.

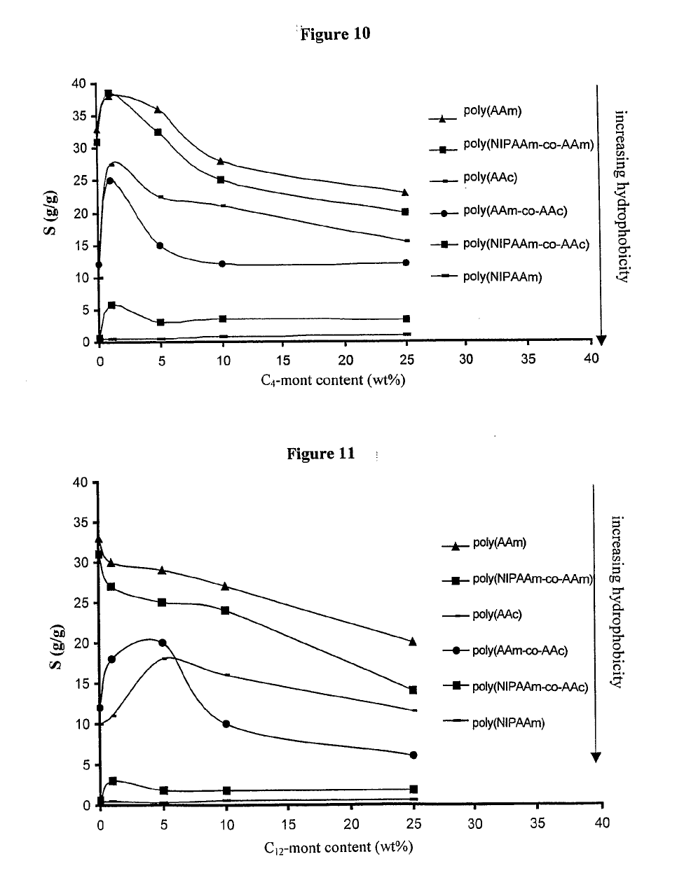

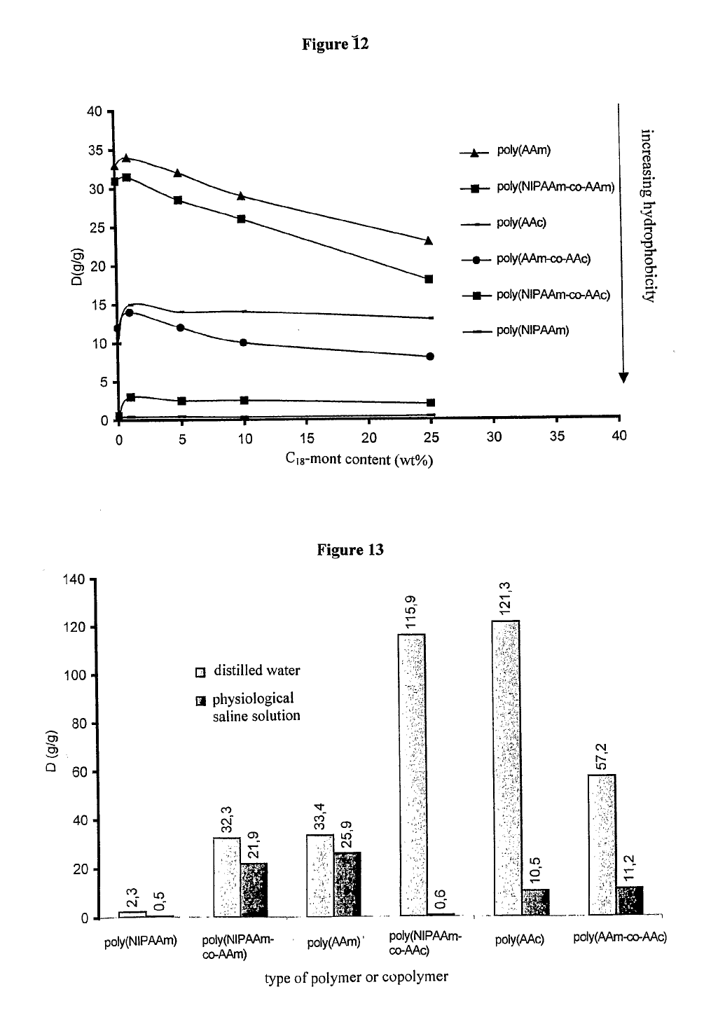

The swelling characteristics of composites containing hydrophobized montmorillonite fillers as a function of filler content were compared. The swelling characteristics of composites with C₄-mont, C₁₂-mont and C₁₈-mont filler are presented in FIGS. 10, 11 and 12, respectively, these figure differs from each other in the quality of the filler material. The conclusions drawn for Na-montmorillonite, namely that low concentrations of fillers improve the swelling characteristics of the samples also hold for these fillers. According to our results, this phenomenon is practically independent of either copolymer or filler type.

Again the extent of swelling is primarily determined by the hydrophilicities of the monomers constituting the copolymer and by the ratio of monomers of different hydrophilicities rather than by the hydrophilicity of the filler: copolymers of identical composition but different filler contents produce curves that run identical courses and there are no great differences between the extents of their swelling. Considering, for example, the swelling of the most extensively swelling sample, the 100% AAm-based composite, it can be established that at any filler content the differences between the extents of swelling of the samples are within 3-7%.

Based on the data in Table 2 it can be established that, in the case of NIPAAm and/or AAm based gels, the more hydrophobic the starting monomer, the more 1 to 5 wt % filler content increases swelling. In the case of NIPAAm and/or AAc based samples the effect is not so evident: the largest difference, 445% is observed in the case of the poly(NIPAAm-co-AAc) sample, whereas the filler increased swelling of pure NIPAAm and AAc based samples by 27% and 180%, respectively. Swelling of AAm and/or AAc based samples is also affected relatively extensively by the presence of the filler.

To sum up, it can be concluded that swelling of composites is significantly affected by filler concentration. FIGS. 9 to 12 reveal that, at relatively low filler concentrations, the extent of swelling can be increased in the case of all nanocomposites studied, as compared to homo- and copolymers without filler. As regards the relationship between swelling values and fillers, it can be concluded that hydrophilic fillers (Na-mont or C₄-mont) increase the swelling of hydrophilic polymers (AAm and AAc), whereas hydrophobic fillers (C₁₂ and C₁₈) mainly affect swelling of the hydrophobic NIPAAm-based homo- and copolymers.

TABLE 2
The effect of fillers on the swelling of the samples

| | Swelling of | Maximal | | Filler | |
|---|---|---|---|---|---|
| | polymer | swelling of | | concentration in | |
| | sample | sample | | the sample at | |
| | without filler | containing filler | Filler | maximal | Difference |
| Polymer sample | [D(g/g)] | [D(g/g)] | quality | swelling | (%)* |
| poly(NIPAAm) | 0.55 | 0.7 | Na-mont | 1 | 27 |
| poly(NIPAAm-co-AAm) | 32 | 26 | C₄-mont | 1 | 13 |
| poly(AAm) | 33 | 38 | C₄-mont | 5 | 15 |
| poly(NIPAAm-co-AAc) | 1.1 | 6 | C₄-mont | 1 | 445 |
| poly(AAc) | 10 | 28 | Na-mont | 1 | 180 |
| poly(AAm-co-AAc) | 11 | 35 | C₄-mont | 1 | 218 |

*Difference (%): the excess swelling due to the presence of the filler, with the swelling of the sample without filler taken as 100%

The Effect of Electrolyte Concentration on the Kinetics of Composite Gels

Since part of our in vitro studies were performed in distilled water, swelling values of the individual samples in distilled water and in physiological saline were compared in order to enable conclusions to be drawn from swelling values obtained in distilled water regarding swelling expected under physiological conditions.

Swelling values of the various homo- and copolymers in distilled water were compared to those in physiological saline in FIG. 13. In both series of experiments the pH of the samples was kept at a constant value of pH=7. The values measured in saline lag behind those measured in distilled water in all samples. The differences measured, however, are different in the case of the individual copolymers: AAm-based gels are the least and NIPAAm- and AAc-based gels are the most sensitive to salt content. When the NIPAAm monomer copolymerized with AAc, the difference is close to 200-fold. Percentage differences between swelling values measured in the two media are also listed in Table 3.

TABLE 3
Comparison of polymer swelling in distilled
water and physiological saline

| | Swelling | | |
|---|---|---|---|
| | in dist. water | Swelling in phys. saline | Difference |
| Polymer/copolymer | D (g/g) | D (g/g) | (%)* |
| 100% NIPAAm | 2.3 | 0.5 | 360.00 |
| 50% NIPAAm-50% | 32.3 | 21.9 | 47.49 |
| AAm | | | |
| 100% AAm | 33.4 | 25.9 | 28.96 |
| 50% NIPAAm-50% | 115.9 | 0.6 | 19216.67 |
| AAc | | | |
| 100% AAc | 121.3 | 10.5 | 1055.24 |
| 50% AAm-50% | 57.2 | 11.2 | 410.71 |
| AAc | | | |

*Difference (%): [(D_{dist.water} − D_{phys.saline})/D_{phys.saline}] * 100

Gel Swelling as a Function of Temperature

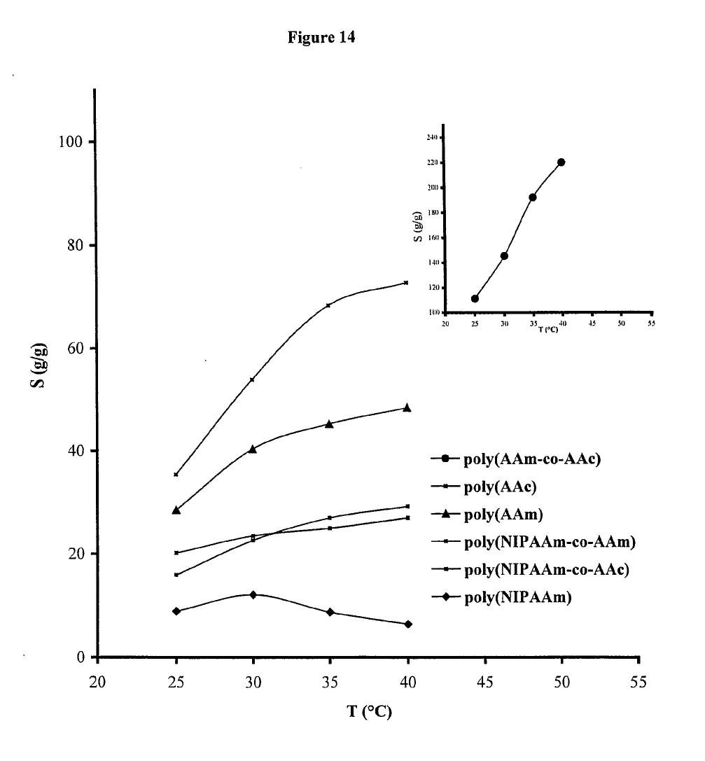

These experiments were carried out in order to enable conclusions to be drawn from swelling measured at room temperature under in vitro conditions to values expectable at body temperature. As shown in FIG. 14, the swelling maximum of thermosensitive poly(NIPAAm) is at 31° C. and at higher temperatures the gel collapses. When the NIPAAm monomer is copolymerized with AAm or AAc. swelling of the samples increases continuously with increasing temperature, i.e. the copolymer does not collapse as would NIPAAm. The hydrophilicity of the gels decreases from the top of the figure down. The slope of the curves increases with hydrophilicity, indicating that the more hydrophilic the gel, the more extensive is swelling elicited by increasing the temperature.

Analysis of the Mechanical Properties of the Gels

Hydrogels are viscoelastic materials, whose mechanical properties can be examined basically by two methods, namely by static and dynamic load tests.

The static method subjects the sample to instantaneous external loading and, maintaining the load for a given time, examines how the material adapts itself to the load as a function of time; then, after withdrawing the load, the time dependence of the relaxation process is studied. Results obtained by this method are the so-called creeping curves describing the time dependence of shear sensitivity, which give information on the elastic and viscous behavior of the sample under static conditions.

In the dynamic method the external load is an oscillatory load with a given frequency and amplitude, therefore this testing method is also called forced oscillation. Since the external load (shear stress or deformation) is time dependent, this also affects the adaptation of the material, the deformation or tension produced by the load.

In typical dynamic tests the frequency dependence of the reaction of the material tested is obtained by keeping the amplitude of the external load (shear stress or deformation) at a constant value and varying the dynamic loading frequency (frequency sweep). The inverse of this test at a constant loading frequency yields the amplitude dependence of the response (stress sweep). The viscoelastic parameters of the material at the time of dynamic loading are the storage modulus (G′, the elastic component of rheological behavior) and the relaxation modulus, or loss modulus (G″, the viscous component of rheological behavior). If the values of these moduli are independent of the frequency or the amplitude in a certain region of the measurement range, the values obtained are characteristic of the mechanical properties of the given material. This range is termed the range of linear viscoelasticity. Parameters characteristic of the material and independent of the loading conditions can only be determined within this range.

Based on the data in the literature, nanocomposites according to the invention are studied using the following procedures:
In the course of static measurements, the samples were exposed to 1 Pa shear stress for 60 sec, the load was then removed and relaxation of the gel was observed for a further 60 sec;
From the dynamic measurement methods available, the frequency dependence of the samples was examined: the frequency was varied between 0.1 Hz and 1 Hz at a constant shear stress of 1 Pa. Thus the storage modulus (G′) and the loss modulus (G″) were determined.

The rheological behavior of swollen gels was studied at 25° C. by oscillation rheometry. The PP20 sensor (measuring head) (diameter 20 mm, parallel-plate geometry) of a Rheotest RS 150 (HAAKE) oscillatory rheometer was used. Disks of about 3 mm thickness were sliced from the swollen gel cylinders using a scalpel; the diameter of the disks corresponded to that of the measuring head. The plate-plate gap was chosen as 2.5 mm.

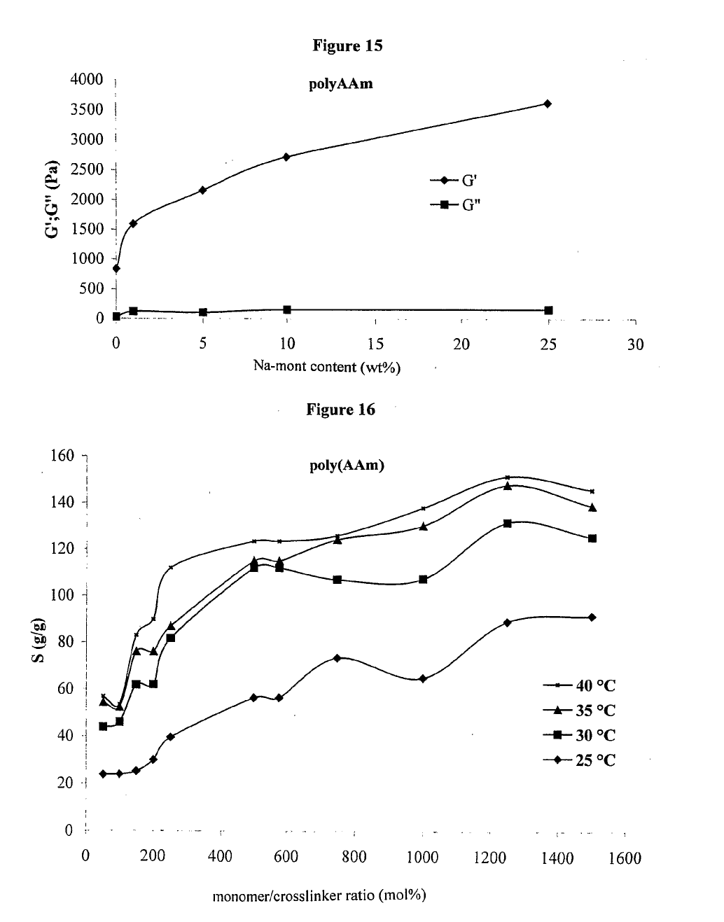

The effect of Na-montmorillonite filler concentration on the mechanical properties of polyAAm is shown in FIG. 15. In the course of the measurement, the frequency applied was varied (0.1-1 Hz) at a constant shear stress (1 Pa), and the storage modulus (G′) characterizing the elastic properties of the sample and the loss modulus (G″) characterizing its viscous properties were measured. It can be established that increasing the filler concentration clearly enhances the elasticity of the sample: the G′ value of the polymer without filler is only 839.44 Pa, whereas that of the gel containing 25 wt % Na-mont exceeds 3600 Pa. The largest increase in G′ is observed in the filler concentration range of 0 to 5 wt %. It is also obvious that G″, a value characteristic of the viscous property of the sample is practically independent of Na-mont content, demonstrating that the mechanical character of these samples is predominantly elastic.

The values of the storage modulus (G′) used for the characterization of the mechanical properties of the gels are listed in Table 4. This number expresses the elastic properties of the samples, thus the higher its value, the more elastic is the gel or composite studied. The data in the table reveal that the value of G′ increases with increasing the filler concentration, i.e. increasing the concentration of filler in the gel increases the elasticity, i.e. the retention of the shape preservation capability of the samples. This holds for practically each filler, irrespective of the quality of the polymer matrix it is dispersed in. Thus, the mechanical properties of composites supplemented with fillers are clearly superior to those of gels without fillers.

TABLE 4
The effect of fillers on the mechanical properties of the samples

| | Monomer | | | |
|---|---|---|---|---|
| | composition | Filler quality (different | Filler concentration | |
| Gel | (mol %) | hydrophilicities) | (wt %) | G′ (Pa) |
| poly(NIPAAm- | 50% NIPAAm- | Na-mont | 0 | 408.64 |
| co-AAm) | 50% AAm | | 1 | 716.15 |
| | | | 5 | 1430.1 |
| | | | 10 | 1388.1 |
| | | | 25 | 2566 |
| poly(AAm) | 100% AAm | Na-mont | 0 | 839.44 |
| | | | 1 | 1596.2 |
| | | | 5 | 2155.8 |
| | | | 10 | 2719.8 |
| | | | 25 | 3625.1 |
| | | C₄-mont | 0 | 839.44 |
| | | | 1 | 1201.3 |
| | | | 5 | 795.5 |
| | | | 10 | 903.56 |
| | | | 25 | 977.63 |
| | | C₁₂-mont | 0 | 839.44 |
| | | | 1 | 1204.8 |
| | | | 5 | 1141.7 |
| | | | 10 | 1770.9 |
| | | | 25 | 2930.4 |
| | | C₁₈-mont | 0 | 839.44 |
| | | | 1 | 791.06 |
| | | | 5 | 758.89 |
| | | | 10 | 962.77 |
| | | | 25 | 1252.73 |
| poly(AAc) | 100% AAc | Na-mont | 0 | 323.28 |
| | | | 1 | 1206.4 |
| | | | 5 | 1587.9 |
| | | | 10 | 1789.9 |
| | | | 25 | 5261.7 |
| | | C₄-mont | 0 | 323.8 |
| | | | 1 | 1486.4 |
| | | | 5 | 977.64 |
| | | | 10 | 1411.8 |
| | | | 25 | 1692.7 |
| | | C₁₂-mont | 0 | 323.28 |
| | | | 1 | 1879.5 |
| | | | 5 | 1415.6 |
| | | | 10 | 1890.7 |
| | | | 25 | 1919.2 |
| | | C₁₈-mont | 0 | 323.28 |
| | | | 1 | 1143.1 |
| | | | 5 | 1164.21 |
| | | | 10 | 1309.8 |
| | | | 25 | 1661.1 |
| poly(AAm-co- | 50% AAm-50% | Na-mont | 0 | 2500 |
| AAc) | AAc | | 1 | 1358.7 |
| | | | 5 | 2818.1 |
| | | | 10 | 5701.9 |
| | | | 25 | 7092.6 |

The Effect of the Monomer/Crosslinker Ratio on the Swelling Characteristics of the Gels

Swelling of AAm-based gels as a function of the monomer/crosslinker (M/C) ratio is presented in FIG. 16. BisAAm was used as crosslinker, and swelling was studied in the temperature range of 25-40° C. in distilled water. The monomer/crosslinker ratio was varied between 50 and 1500. As shown in the figure, the more the M/C ratio is increased—i.e. the more the number of crosslinks in the sample are decreased—, the more the swelling of the gels is enhanced. Swelling definitely increases with increasing temperature.

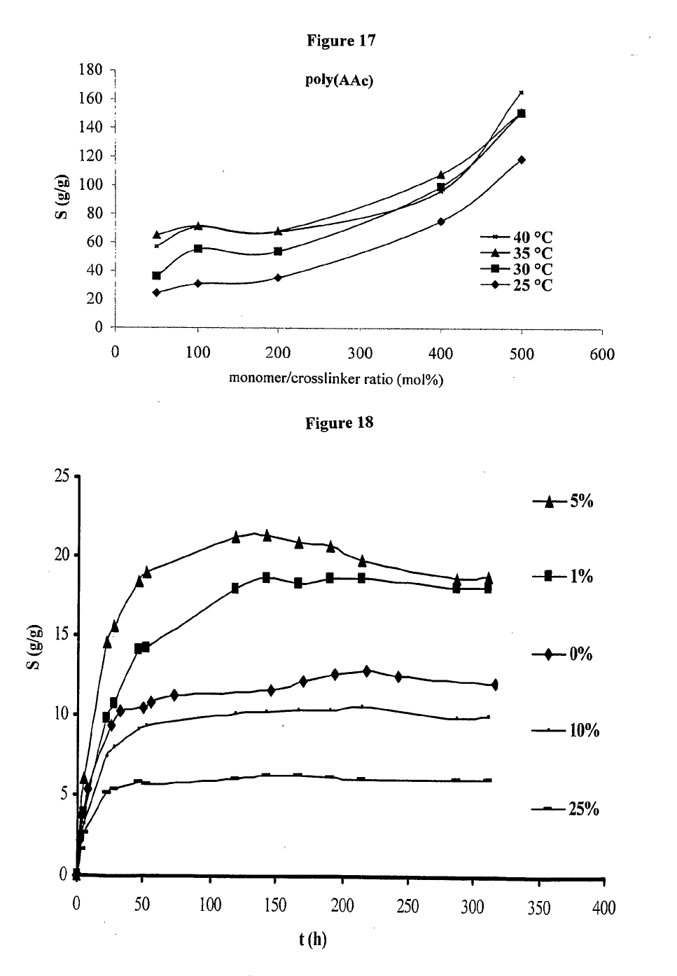

Swelling of AAc-based gels as a function of the monomer/crosslinker (M/C) ratio is shown in FIG. 17. In the case of these gels increasing the M/C ratio resulted in enhanced swelling. The MIC ratio was varied from 50 to 500, and gel swelling is seen to increase with decreasing the number of crosslinks in a linear fashion in this range.

Comparison of FIGS. 16 and 17 reveals that swelling of the hydrophilic AAm and AAc based gels expressly increases with decreasing the number of crosslinks and with increasing the temperature.

The Kinetics of the Swelling of Polymer Nanocomposites

In view of the future utilization of the samples, it is an important expectation that the rate of swelling could be controlled; the swelling of the samples was therefore examined as a function of time. FIG. 18 shows the time dependence of the swelling of poly(AAm-co-AAc) hydrogel samples containing various amounts of C₁₂-montmorillonite. The curves follow similar courses and their initial slopes are also identical, it can thus be established that the fillers do not affect the rate of swelling. Irrespective of filler concentration, the gels reached the equilibrium swelling values corresponding to the given conditions (36.5° C., physiological saline) within 50-75 hours. This holds for practically all analyzed polymers and copolymers supplemented with fillers. Again, however, relatively low filler contents (1 to 5 wt %) are seen to bring about more extensive swelling than either the absence of fillers or their presence in relatively high concentrations (10 to 25 wt %).

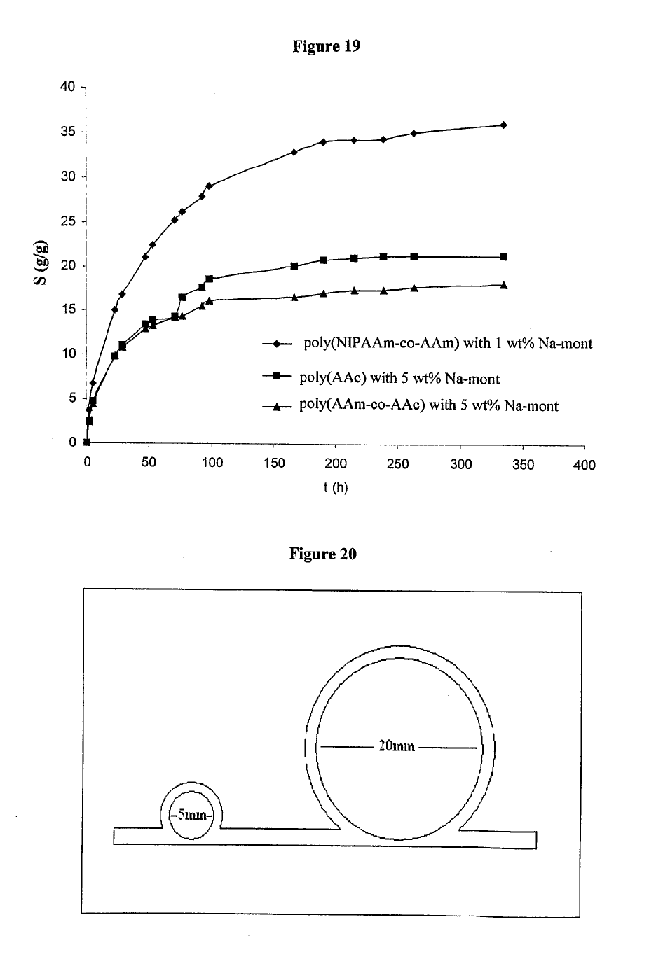

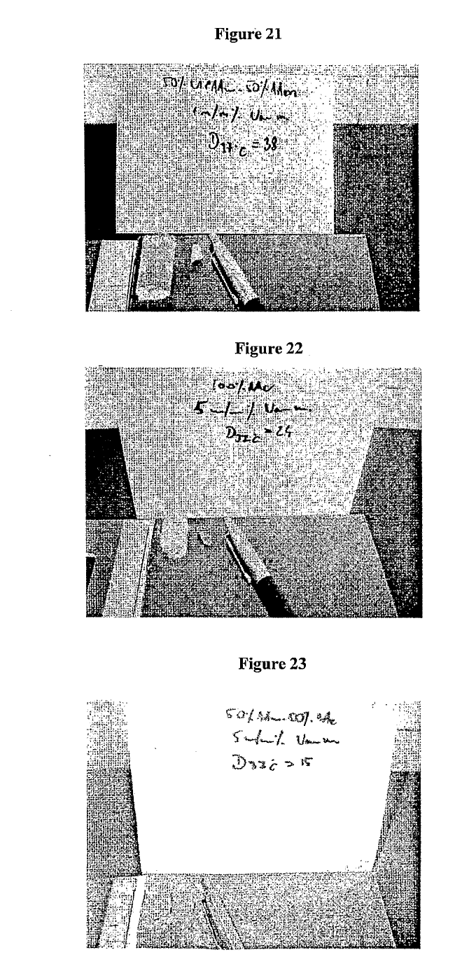

The kinetics of the in vitro expansion of the implanted polymers presented in FIGS. 21 to 23 is shown in FIG. 19. The figure reveals that, under in vitro conditions, swelling is essentially completed within 2 to 3 days. The biological results presented below, however, suggest that advantageously, this process is considerably slower under in vivo conditions, because the expanding tissues exert a force of opposite direction on the swelling nanocomposite hydrogel, and the volume that is to accommodate swelling is created by gradual tissue expansion. In addition, the rate of expansion can be controlled by enclosing the expander in a suitable semipermeable membrane, whose permeability determines the influx rate of the fluid that swells the hydrogel.

In Vivo Studies on the Polymer Gel Composites According to the Invention

Utilization of the Gels for Skin Expansion

The experiments were carried out using Wistar rats, each with approximately 250 g body mass. The rats were kept under appropriate, constant conditions regarding both food and fluid supply.

In the course of the experiments, polymer hydrogel nanocomposite expanders of fixed size, in dried state were implanted under the skin on the back of the rats through small incisions; the wound was then closed. Based on the maximal volume previously achieved in swelling experiments, the ideal location of the hydrogel implant was calculated, taking into account the expected final swollen volume as well as the location of the lesion to be supplemented. The ideal location is about 1 cm from the latter. Thus, in the course of volume increase it is the intact skin that is expanded. Swelling was checked on a daily basis by both photography and recording the change in size.

The expander developed in our laboratory expands to about 40 times its original volume, as shown in FIG. 20. The size of the expanded skin is described by the relationship D·π/2, i.e. a 150% expansion is achieved (considering a cylinder with a diameter of 2 cm, a 3 cm length of expanded skin is gained).

In the course of the experiments, material of standard size was always used. The initial size of the dried gel was 5 mm×10 mm. After fluid absorption the volume increased about 40-fold, resulting in a final size of 20 mm×30 mm (FIGS. 21, 22 and 23).

In in vivo studies maximal volume was attained by the 3^{rd} week of expansion.

The results of in vivo studies are presented below in Table 5.

TABLE 5
Results of in vivo studies

| | | | | | Maximal | | | | | |
|---|---|---|---|---|---|---|---|---|---|---|
| | | Monomer/ | Filler | | swelling under | | | | | |
| Ser | Monomer composition | cross-linker | | quantity | physiological | Amounts normalized to 1 g of dried sample (mg) | | | | |
| No | (n/n %) | ratio | quality | (wt %) | conditions | NIPAAm | AAm | AAc | BisAAm | filler |
| 1 | 100% AAm | 750 | 0 | 0 | 31 | 0 | 997.1 | 0 | 2.88 | 0 |
| 2 | 100% AAm | 1300 | 0 | 0 | 36 | 0 | 998.3 | 0 | 1.66 | 0 |
| 3 | 80-20% NIPAAm-AAm | 500 | 0 | 0 | 12 | 861.7 | 135 | 0 | 2.9 | 0 |
| 4 | 100% AAc | 1000 | 0 | 0 | 23 | 0 | 0 | 997.8 | 2.135 | 0 |
| 5 | 100% AAc | 1500 | 0 | 0 | 34 | 0 | 0 | 998.5 | 1.424 | 0 |
| 6 | 80-20% NIPAAm-AAm | 200 | C18 | 1 | 10 | | | | | |
| 7 | 80-20% NIPAAm-AAm | 500 | C18 | 1 | 12 | | | | | |
| 8 | 80-20% NIPAAm-AAm | 200 | Na-mont | 0.1 | 14 | | | | | |
| 9 | 80-20% NIPAAm-AAm | 500 | Na-mont | 0.2 | 22 | | | | | |
| 10 | 50-50% NIPAAm-AAm | 200 | 0 | 0 | 32 | 609.1 | 382.6 | 0 | 8.3 | 0 |
| 11 | 50-50% NIPAAm-AAm | 200 | Na-mont | 1 | 38 | 603 | 378.8 | 0 | 8.2 | 10 |
| 12 | 50-50% NIPAAm-AAm | 200 | Na-mont | 5 | 32 | 578.8 | 363.5 | 0 | 7.88 | 50 |
| 13 | 50-50% NIPAAm-AAm | 200 | Na-mont | 10 | 28 | 548.2 | 344.3 | 0 | 7.47 | 100 |
| 14 | 100% AAm | 200 | 0 | 0 | 33 | 0 | 989.3 | 0 | 10.73 | 0 |
| 15 | 100% AAm | 200 | Na-mont | 1 | 29 | | | | | |
| 16 | 100% AAm | 200 | Na-mont | 5 | 36 | | | | | |
| 17 | 100% AAm | 200 | Na-mont | 10 | 29 | | | | | |
| 18 | 100% AAm | 200 | Na-mont | 25 | 24 | | | | | |
| 19 | 100% AAc | 200 | Na-mont | 5 | 24 | 0 | 0 | 939.9 | 10.05 | 50 |
| 20 | 100% AAc | 200 | Na-mont | 10 | 22 | 0 | 0 | 890.5 | 9.53 | 100 |
| 21 | 100% AAc | 200 | Na-mont | 25 | 16 | 0 | 0 | 742 | 7.94 | 250 |
| 22 | 50-50% AAm-AAc | 200 | Na-mont | 1 | 24 | 0 | 486.37 | 493 | 10.5 | 10 |
| 23 | 50-50% AAm-AAc | 200 | Na-mont | 5 | 15 | | 466.72 | 473.16 | 10.12 | 50 |

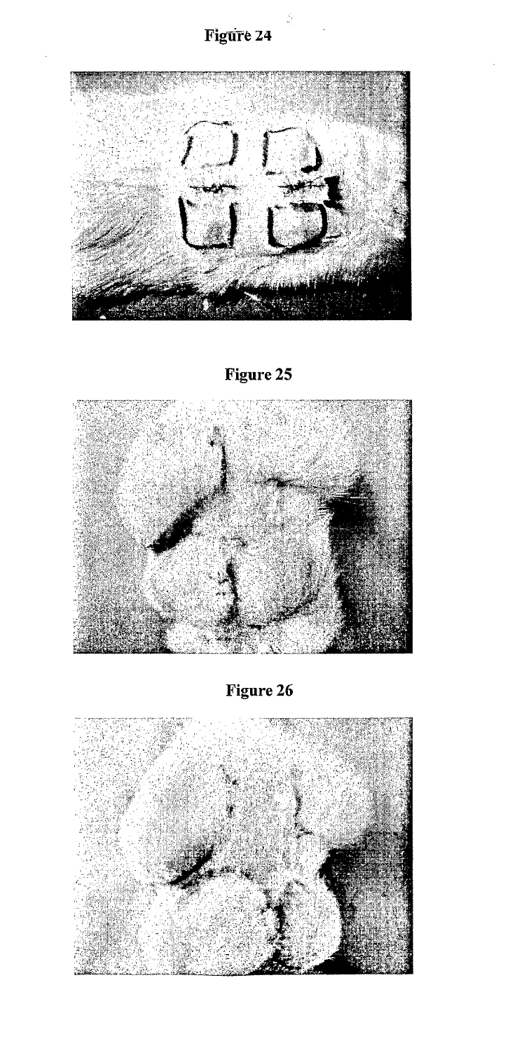

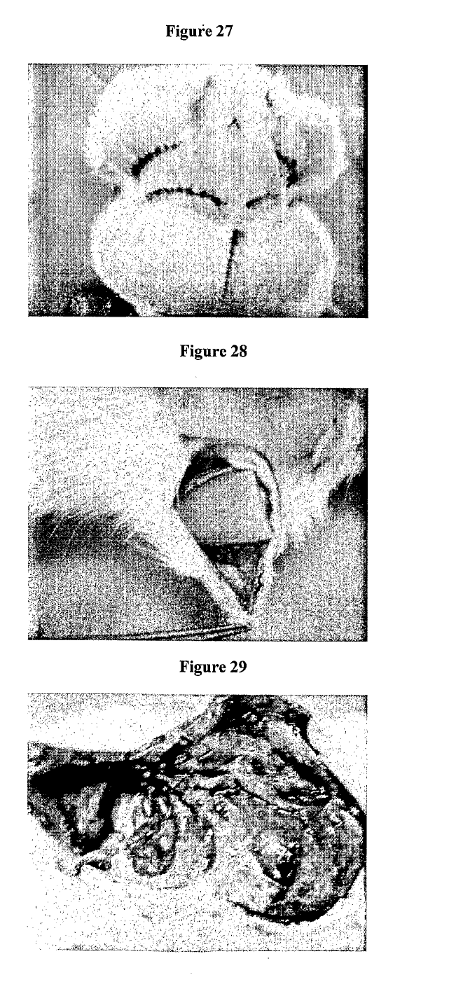

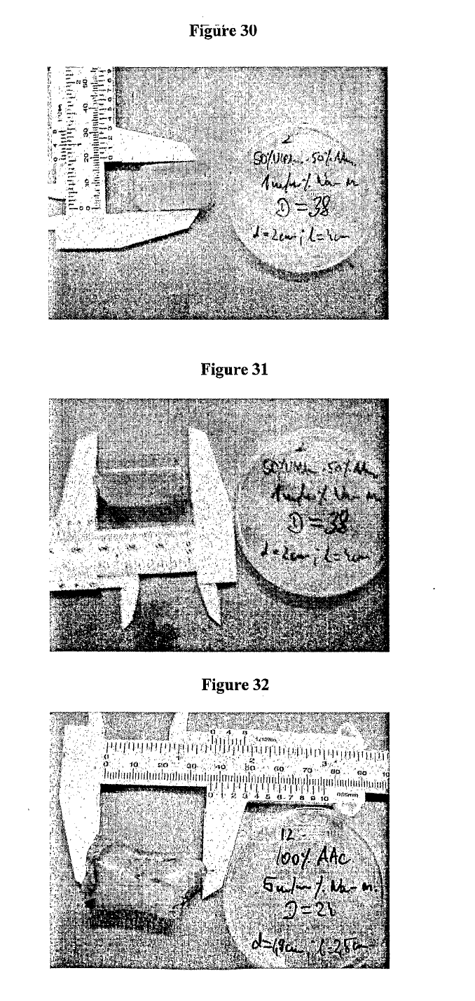

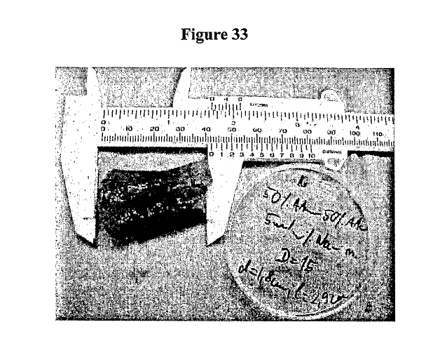

FIG. 24 was taken after implantation of the samples into rats. FIGS. 25 to 27 present the process of swelling under in vivo conditions. FIGS. 28 to 33 were taken after excision of the samples.

### SUMMARY

Based on the above experimental results it can be established that nanocomposites composed of hydrogels synthetized by copolymerization of N-isopropylacrylamide, acrylamide and/or acrylic acid monomers supplemented with hydrophobized layer silicates, constituting the object of our invention are well applicable to tissue expansion for the purpose of obtaining skin production. By the evidence of our studies the nanocomposites implanted under the skin retained their chemical stability throughout the period studied; the kinetics of swelling is satisfactory and, due to their mechanical and geometrical stability, they ensure proportional skin expansion. The volume expansion of the filler-containing polymer gel according to the invention is significantly higher than that of other similar materials described in the technical literature: it amounts to about 40 times its original volume.

1. An osmotically active nanocomposite for use in expanding live skin comprising a hydrogel synthesized by polymerization of N-isopropylacrylamide and/or acrylamide and/or acrylic acid monomers and as a crosslinker N,N-methylenebisacrylamide; and a layer of silicate filler, which hydrogel comprises
0 to 90 mol % of N-isopropylacrylamide monomer and 100 to 10 mol % of acrylamide monomer, or
0 to 30 mol % of N-isopropylacrylamide monomer and 100 to 70 mol % of acrylic acid monomer, or
0 to 100 mol % of acrylamide monomer and 100 to 0 mol % of acrylic acid monomer; and
the ratio of the crosslinker to the total sum of N-isopropylacrylamide monomer and acrylamide monomer and acrylic acid monomer ranges from 1:50 to 1:1500, and
the concentration of the layer silicate filter is 0.1 to 10 wt % relative to the total mass of the nanocomposite.

2. (canceled)

3. (canceled)

4. The osmotically active nanocomposite for use in expanding live skin according to claim 1, which comprises Na-montmorillonite or montmorillonite organophilized by alkylamines having 4 to 18 carbon atoms as filler.

5. The osmotically active nanocomposite for use in expanding live skin according to claim 1 which swells to 15-40 times its original volume under physiological conditions.

6. The osmotically active nanocomposite for use in expanding live skin according to claim 1 wherein the live skin obtained is used for supplementing skin deficiencies.

7. An osmotically active tissue expander which is prepared from the nanocomposite according to claim 1.

8. Use of the expander according to claim 7 for expanding live skin.

9. The use according to claim 8 for obtaining live skin suitable for supplementing skin deficiency, for example in the case of burns or in the course of the correction of congenital disorders.

10. (canceled)
